(19) United States
(12) Patent Application Publication
(10) Pub. No.: US 2005/0184116 A1
(43) Pub. Date: Aug. 25, 2005

(54) Extendable rack

(76) Inventor: Duane Jury, Silverton, OR (US)

Correspondence Address: DAVID W.C. CHEN, P.O. BOX 3041, SEATTLE, WA, 98114-3041, US

(21) Appl. No.: 10/783,319

(22) Filed: Feb. 20, 2004

(51) Int. Cl. A61M 5/00; B60R 7/00; B60R 9/00

(52) U.S. Cl. 224/311; 224/405

(57) ABSTRACT

An extendable rack mountable to a vehicle. The extendable rack may be extended to allow easier access to items supported thereon.

### BACKGROUND OF THE INVENTION

1. Field of the Invention

The present invention relates generally to racks, and more particularly to an extendable rack adapted for mounting to a vehicle.

2. Description of Related Art

It is familiar practice to mount utility racks to open-bed pickup trucks and other vehicles and to secure items to the utility racks during transport. This allows secured items to be inspected and retrieved more easily and rapidly than if they were merely stacked and can also reduce the risk of damage to the items. Some commonly used utility racks on pick-up trucks have elevated supports bars that extend laterally across, or longitudinally parallel to a longitudinal axis of the vehicle or its bed. Items are suspended or secured to the elevated support bars, either above or below them, to prevent the items from cluttering the floor of the vehicle or for safekeeping. However, items elevated in such a fashion can be out of the easy reach for some users. For example, retrieving an item suspended from an elevated support in a pick-up truck can require one to climb into the bed of the pick-up truck before reaching the item. For some users, this physical exertion is difficult or even painful, such as for those having back problems or other physical limitations. Also, climbing into a pick-up bed or onto other raised surfaces requires expenditure of time and effort.

There is a need for a rack that makes items suspended from elevated supports more easily accessible to users with physical limitations and to alleviate time and physical exertion spent climbing into and out of a vehicle or truck bed.

### BRIEF SUMMARY OF THE INVENTION

The present invention resides in an extendable rack. There is a retainer that can be tubular in shape, with a slidable member slidably attached to the retainer. The slidable member can have apertures with integral notches or hooks attached thereto. The retainer can also have a slit that extends longitudinally along a portion of the retainer. In some embodiments the integral notches can be aligned with the slit and in other embodiments the hooks can be aligned with the slit.

In further embodiments of the invention, the extendable rack is attached to a utility rack of a vehicle, such as an open bed pickup truck, or to a surface within a vehicle, such as a van. The slidable member is slidable such that a portion of the slidable member can be extended beyond a perimeter of the vehicle, such as, for example, an end portion of the vehicle.

Other features and advantages of the invention will become apparent from the following detailed description and the accompanying drawings.

### BRIEF DESCRIPTION OF THE SEVERAL VIEWS OF THE DRAWING

FIG. 1 is a cross-sectional view illustrating an embodiment of the present invention showing a slidable member coaxially disposed within a retainer with a hook connected to a reinforcement insert within the slidable member.

FIG. 2 is a cross-sectional view of the retainer and slidable member of FIG. 1, without a reinforcement insert or a hook.

FIG. 3 is a cross-sectional view illustrating the retainer of FIG. 2 without the slidable member disposed therein.

FIG. 4 is a perspective view illustrating the slidable member and retainer of FIG. 1 with the slidable member partially inserted within the retainer.

FIG. 5 is a perspective view illustrating the retainer of FIG. 3.

FIG. 6 is a perspective view illustrating the slidable member of FIG. 1 and FIG. 4 showing a plurality of hooks coupled thereto.

FIG. 7 is a cross-sectional view illustrating an embodiment of the present invention showing a retainer that is connected to twin mounting strips by a semicircular support member.

FIG. 8A is a perspective view of the embodiment of the retainer of FIG. 7.

FIG. 8B is a perspective view of another embodiment of the retainer.

FIG. 9 is a perspective view illustrating an embodiment of the slidable member wherein it is a tube having a plurality of circular apertures with integral notches, the circular apertures being spaced apart longitudinally on the slidable member.

FIG. 10 is a perspective view of an embodiment of the present invention comprising the retainer of FIG. 5 and the slidable member of FIG. 9. The slidable member is shown partially inserted within the retainer with an end portion of the slidable member extending outward from an end of the retainer.

FIG. 11 is a partial perspective view of a portion of the slidable member of FIG. 9, with the slidable member partially rotated about its longitudinal axis in the direction of arrow “A” from its position in FIG. 9 to face the circular apertures laterally outward, and with a cord and retaining ball assembly shown proximate the circular aperture positioned to be inserted into the circular aperture and notch in the direction of arrow “B”.

FIG. 12A is a partial perspective view illustrating the slidable member of FIG. 11 coaxially disposed within the retainer of FIG. 5 and partially extended therefrom, and with a plurality of cord and ball assemblies inserted into the slidable member with cords extending through a slit in the retainer.

FIG. 12B is a cross-sectional view of the embodiment of the invention illustrated in FIG. 12A.

FIG. 13 is a perspective view of an embodiment of the present invention showing the retainer and slidable member of FIG. 4 mounted on a utility rack of an open-bed pickup truck.

FIG. 14 is a perspective view of an embodiment of the present invention as applied to a van, showing the retainer and slidable member of FIG. 4 mounted on the ceiling of a utility van.

### DETAILED DESCRIPTION OF THE INVENTION

In the following description, certain specific details are set forth in order to provide a thorough understanding of various embodiments of the invention. However, upon reviewing this disclosure one skilled in the art will understand that the invention may be practiced without many of these details. In other instances, well-known structures associated with pick-up trucks and utility racks used therewith have not been described in detail to avoid unnecessarily obscuring the descriptions of the embodiments of the invention.

Certain terms in the following description related to spatial orientation such as “left” and “right,” “top” and “bottom,” “over,” “under” and “around” are only intended to describe the position or orientation of elements in relation to the figures in which they are illustrated, unless the context indicates otherwise.

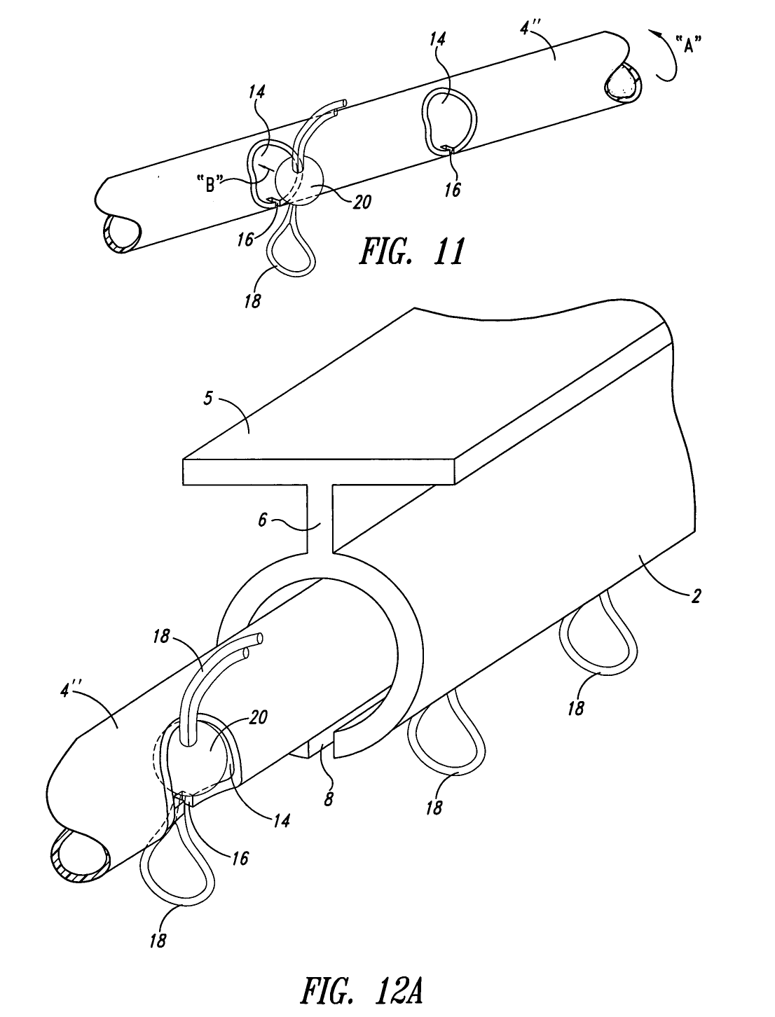

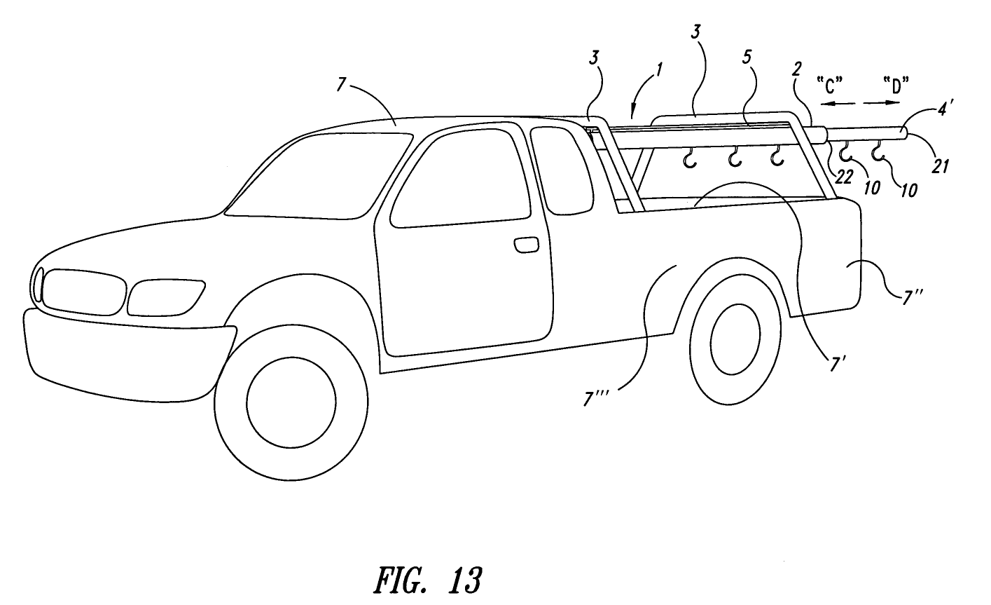

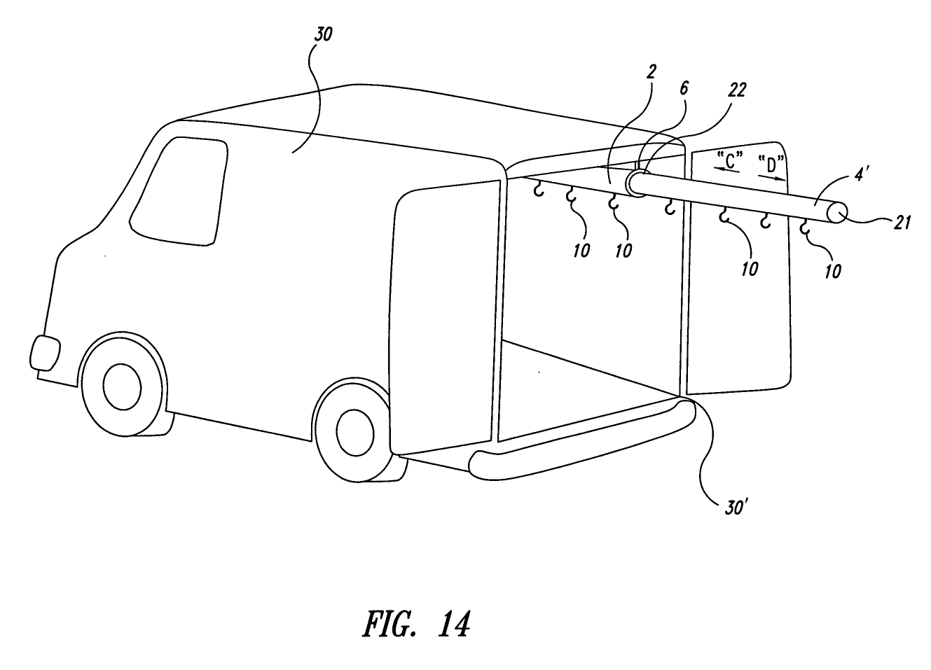

As best seen in FIGS. 4, 10 and 13, some embodiments of the present invention comprise an extendable rack 1 with a retainer 2 and slidable member 4′, 4″. The extendable rack 1 can be suitable for mounting to a utility rack 3 of an open-bed pick-up truck 7 having an open-bed 7′ and a rear portion 7″. See FIG. 13. The extendable rack 1 can also be suitable for mounting to a ceiling of a van 30, or utility van, having a rear portion 30′, as best seen in FIG. 14. The slidable member 4′, 4″ can have holding elements (examples of holding elements include hooks 10, See FIG. 13 and FIG. 14, or apertures 14, See FIG. 10) disposed thereon from which items can be suspended or held in place. A user can selectively retract or extend the extendable rack 1 by sliding the slidable member 4′ relative to the retainer 2 in the directions shown by arrows “C” and “D” in FIG. 13 and FIG. 14. Sliding the slidable member 4′ away from the retainer 2 in the direction of arrow “D” extends the extendable rack 1 and sliding the slidable member toward the retainer 2 in the direction of arrow “C” retracts the extendable rack 1.

When the extendable rack 1 is extended, at least a portion of the slidable member 4′ can be positioned beyond the open-bed 7′ and rear portion 7″ of the pickup truck 7, or rear portion 30′ of the van 30 so that a user can more easily retrieve items suspend from or held by holding elements 10, 14 of the slidable member 4′ while standing behind the pickup truck 7 or van 30. Conversely, when the slidable member 4′ is retracted, it can be slid in a direction toward the retainer 2 until an end portion 21 of the slidable member 4′, 4″ is nearer to, or substantially aligned with an end portion 22 of the retainer 2.

The slidable member 4′, 4″ and retainer 2 may be any of a variety of shapes, such as the tube shaped structures shown in FIGS. 4 and 10 that can be coaxially disposed within one another. Moreover, the retainer 2 can be connected to a vertical support strip 6, which, in turn, can be connected to a mounting strip 5. The mounting strip 5 can be coupled to the utility rack 3 of a vehicle as shown in FIG. 13 or another surface of the vehicle, such as the ceiling portion of a van 30 as shown in FIG. 14. As will be appreciated by one skilled in the art after reviewing the present disclosure, the mounting strip 5 can be attached to the utility rack 3 or other surface by any of a variety of elements including, without limitation, common structures and methods such as screws, pins, straps, nails, welds, adhesives or bolts.

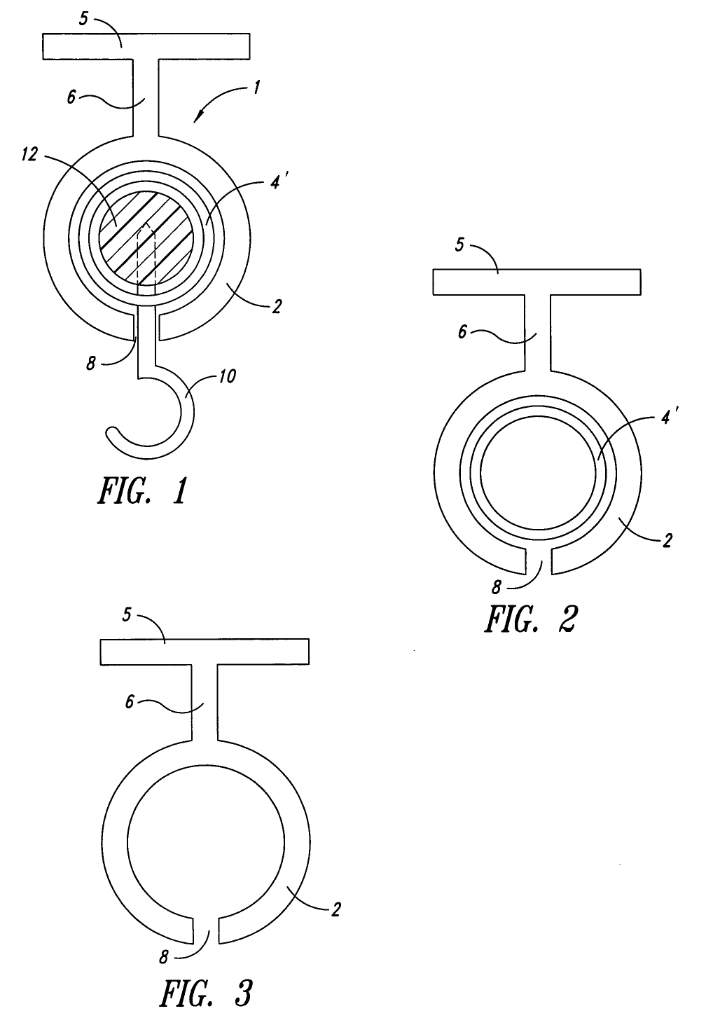

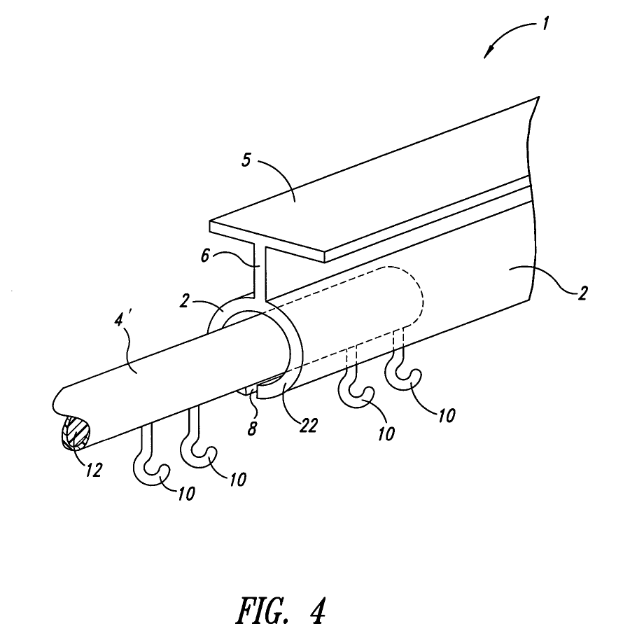

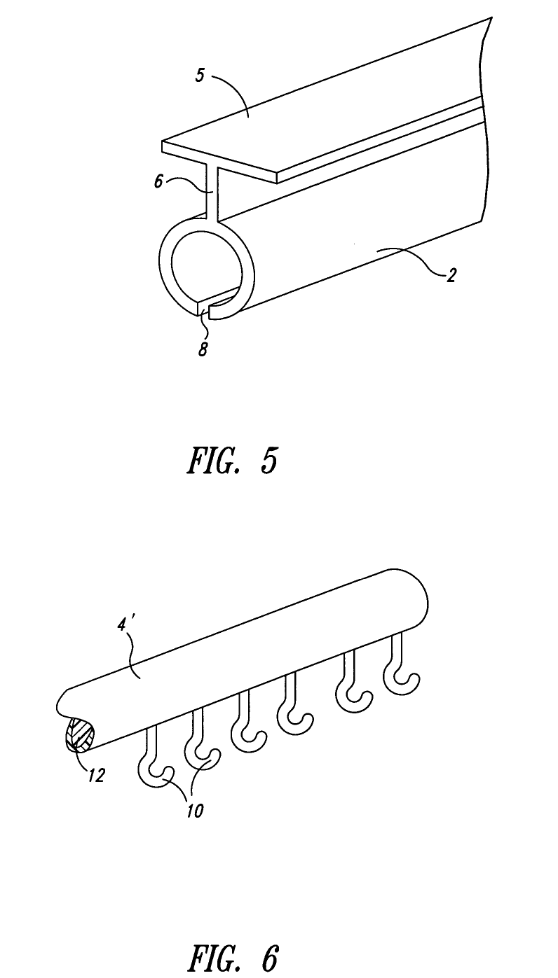

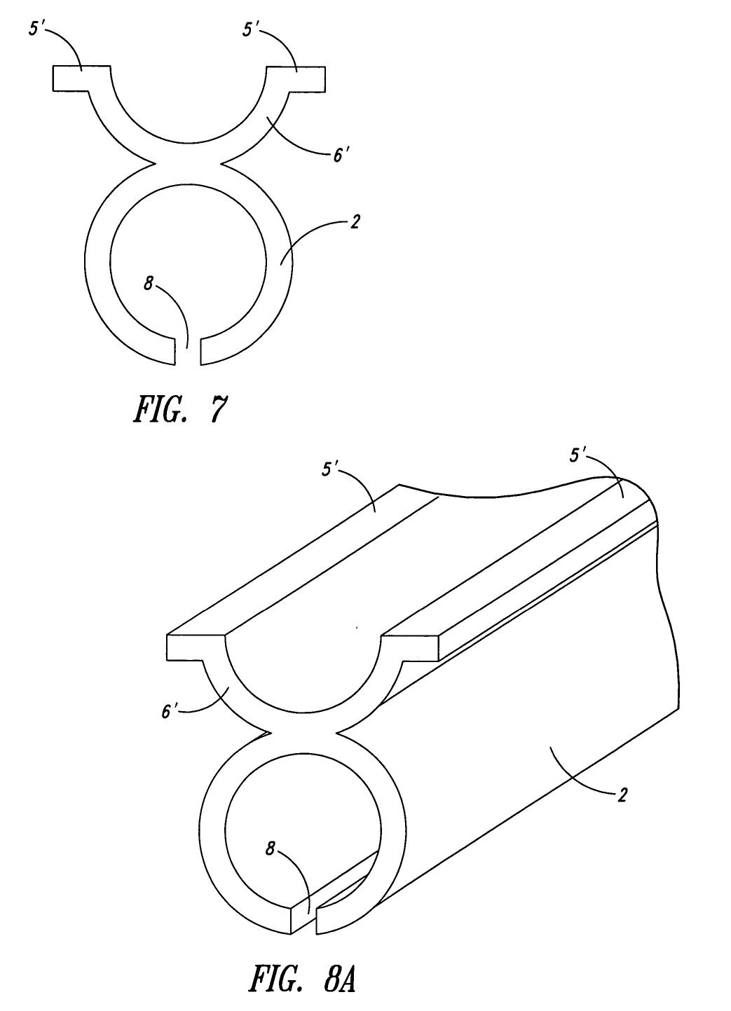

In some embodiments, such as those shown in FIGS. 1, 4, 6 and 13 the slidable member 4′ is provided with hooks 10 to suspend items therefrom. As best seen in FIGS. 1 and 4 when the slidable member 4′ is coaxially disposed within the retainer 2, the hooks 10 can pass through a slit 8 that runs along the length of the retainer 2. The slit 8 can also be seen in FIG. 5 showing the retainer 2 with the slidable member 4′ removed. When the slidable member 4′ is extended or retracted from the retainer 2, the hooks 10 are unobstructed in longitudinal movement along the axis of the retainer because of the passageway provided by the slit 8.

In other embodiments of the invention, holding elements other than hooks 10 may be affixed to the slidable member 4′ for suspending items therefrom. Such apparatus can include, without limitation, rings, screw-eyes, straps, pins, loops of material, and various other elements commonly used to suspend or hold items on racks, as will be appreciated by one skilled in the art after reviewing this disclosure.

In the embodiments of the invention having hooks 10, the slidable member 4′ may be tubular and provided with a reinforcement insert 12. See FIGS. 1 and 4. The reinforcement insert 12 can be a single piece insertable coaxially into the slidable member 4′. The reinforcement insert 12 can be incorporated into embodiments of the invention comprising a light weight slidable member 4′. For example, the slidable member 4′ can comprise polyvinyl chloride plastic while the reinforcement insert 12 can comprise a synthetic mixture of waste wood and recycled plastic such as, without limitation, TREX decking material. As will be appreciated by one skilled in the art after reviewing the present disclosure, a wide variety of other materials may also be used to manufacture the various components of the present invention.

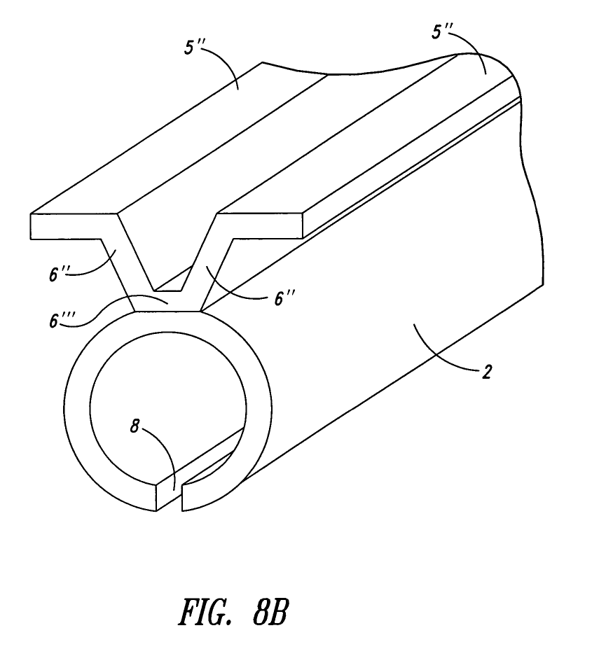

In another embodiment of the present invention, the retainer 2 is connected to two mounting strips 5′ by a semicircular support members 6′, as shown in FIGS. 7 and 8A. Also, another embodiment of the retainer 2, best seen in FIG. 8B, includes mounting strips 5″ with inwardly extending support members 6″ that join at a base 6′″. The mounting strips 5′ and 5″ are attachable to a surface by substantially similar structures and methods as the mounting strip 5.

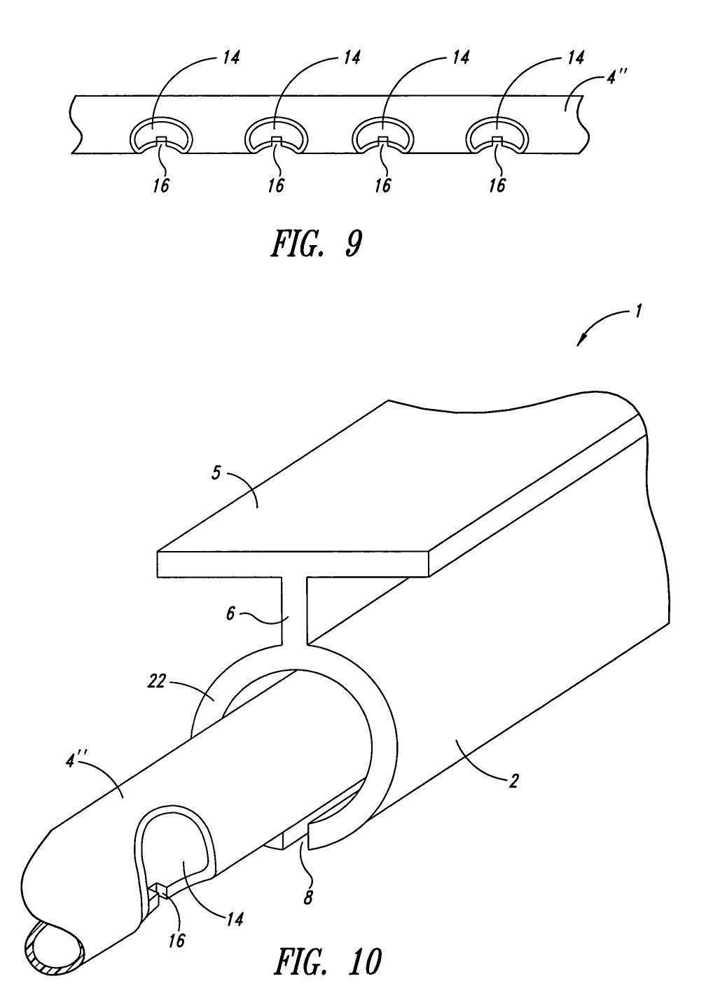

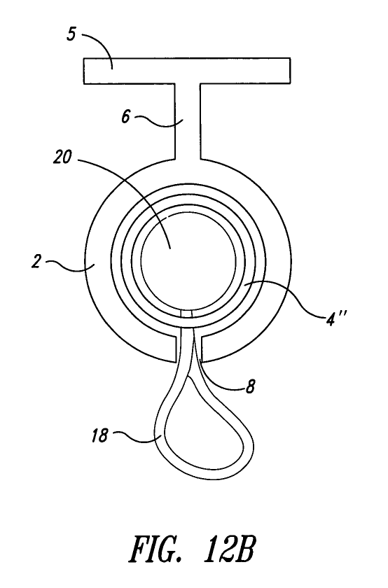

In yet further embodiments, other holding elements are provided for the slidable member 4″, such as circular apertures 14 that incorporate integral notches 16, as best seen in FIGS. 9 and 10. The circular apertures 8 in the slidable member 4″ are suitable for use with an assembly comprising a cord 18, such as a bungee cord, looped through a retaining ball 20. See FIG. 11. When the retaining ball 20 of such an assembly is inserted though one of the slidable member's 4″ circular apertures 14, the cord 18 can be threaded through the corresponding integral notch 16. See FIG. 12A. The slidable member 4″ can then be slid into the retainer 2 so that the cord 18 hangs or protrudes downward through the slit 8 in the retainer 2. In this configuration, items may be hung from the cord 18 while the slidable member 4″ is disposed within the retainer 2. A plurality of cord 18 and retaining ball 20 assemblies can be held in the slidable member 4″ within the retainer 2 in this manner. Because the notches 16 are alignable to coincide with the retainer's 2 slit 8, the protruding portion of the cord 18 can hang down through slit 8 of the retainer 2 and avoid interfering with the extension or retraction of the slidable member 4″.

As will be appreciated by one skilled in the art after reviewing the present disclosure, in some embodiments of the invention, apparatus other than mounting strips 5, 5′, 5″ may be affixed to the retainer 2 for the purpose of mounting the retainer 2 to a utility rack or other surface. Also, the mounting strip 5, 5′,5″ and such other apparatus may be coupled to a utility rack 3 or other surface by a variety of methods, such as, without limitation, welding, bolting, screwing or adhesion.

Some embodiments of the invention include retracted lengths of between four to eight feet, between four to twelve feet, between eight to fourteen feet, and between ten to twenty feet as may be appropriate for the corresponding utility rack 3 or other surface or vehicle to which they are mounted. Other embodiments of the invention may have lengths between fewer than four feet to more than twenty feet.

Some embodiments of the invention may be mounted to a vehicle's utility rack or another surface of the vehicle laterally, longitudinally, or in other orientations. For example, in a lateral orientation, when the extendable rack is extended, the slidable member 4′, 4″ may be positioned beyond a side portion 7′″ of the pickup truck 7 rather than the rear portion 7″.

Also, as will be appreciated by one skilled in the art after reviewing the present disclosure, some embodiments of the invention may incorporate elements for locking or fixing the slidable member 4′, 4″ at full, partial, or zero extension, relative to the retainer 2, such as any of a variety of available devices for locking slidable coaxial tubes in place relative to one another. Examples of such locking elements include, without limitation, a pin insertable through apertures in both the slidable member 4′, 4″ and retainer 2′.

Although specific embodiments and examples of the invention have been described supra for illustrative purposes, various equivalent modifications can be made without departing from the spirit and scope of the invention, as will be recognized by those skilled in the relevant art after reviewing the present disclosure. The various embodiments described can be combined to provide further embodiments. The described devices and methods can omit some elements or acts, can add other elements or acts, or can combine the elements or execute the acts in a different order than that illustrated, to achieve various advantages of the invention. These and other changes can be made to the invention in light of the above detailed description.

In general, in the following claims, the terms used should not be construed to limit the invention to the specific embodiments disclosed in the specification. Accordingly, the invention is not limited by the disclosure, but instead its scope is determined entirely by the following claims.

1. An extendable rack coupled to a vehicle, the extendable rack comprising:
a retainer fixedly attached to the vehicle; and
a slidable member, slidably attached to the retainer such that sliding the slidable member in a direction away from the retainer extends the extendable rack and sliding the slidable member in a direction toward the retainer retracts the extendable rack, said slidable member having a plurality of holding elements for holding items on the extendable rack.

2. The extendable rack of claim 1 wherein the retainer is fixedly attached to the vehicle by being mounted against one of a utility rack coupled to the vehicle and a ceiling portion of the vehicle.

3. The extendable rack of claim 2 wherein at least a portion of the slidable member having holding elements can be positioned beyond a rear portion of the vehicle when the extendable rack is extended.

4. The extendable rack of claim 3 wherein the retainer and slidable member are tubular in shape and coaxially disposed with respect to one another.

5. The extendable rack of claim 4 wherein at least one of the holding elements is a hook.

6. The extendable rack of claim 4 wherein at least one of the holding elements is an aperture configured for receiving a retaining ball attached to a cord.

7. The extendable rack of claim 6 further comprising a notch integral to the aperture.

8. The extendable rack of claim 4 further comprising a reinforcement insert coaxially disposed within the slidable member.

9. The extendable rack of claim 8 wherein the slidable member is comprised of polyvinyl chloride plastic.

10. The extendable rack of claim 8 wherein the reinforcement insert is comprised of wood and plastic.

11. An extendable rack comprising:
a tubular shaped retainer having a slit extending longitudinally along a length of the retainer; and
a slidable member with at least a portion of the slidable member coaxially disposed within the retainer, the slidable member having at least one of a circular aperture having an integral notch configured for receiving a retaining ball and a hook attached to the slidable member.

12. The extendable rack of claim 11 further comprising a mounting strip for mounting the extendable rack to a surface.

13. The extendable rack of claim 12 further comprising a support strip coupled between the mounting strip and the retainer.

14. The extendable rack of claim 11 further comprising a retaining ball and cord assembly, said retaining ball and cord assembly being capable of being attached to the slidable member by inserting the retaining ball within the circular aperture and allowing the cord to protrude through the integral notch.

15. A method of hanging items on a rack comprising:
providing a tubular shaped retainer having a slit extending longitudinally along a length of the retainer;
providing a tubular shaped slidable member with at least a portion of the slidable member coaxially disposed within the retainer, the slidable member having at least one of a circular aperture and a hook attached to the slidable member with a portion of the hook aligned with said slit in the retainer;
sliding the slidable member with respect to the retainer; and
banging at least one item on the slidable member.

16. The method of claim 15 wherein there is a mounting strip coupled to the retainer.

17. The method of claim 16 wherein there is a support strip disposed between the mounting strip and the retainer.

18. The method of claim 15 wherein there is a reinforcement insert coaxially disposed within the retainer.

19. The method of claim 18 wherein the reinforcement member comprises wood.

20. The method of claim 19 wherein the reinforcement member further comprises plastic.
